(19) United States
(12) Patent Application Publication
(10) Pub. No.: US 2020/0138369 A1
(43) Pub. Date: May 7, 2020

(54) FATIGUE RECOVERY SUPPORT APPARATUS

(71) Applicants: Murata Manufacturing Co., Ltd., Nagaokakyo-shi (JP); The University of Electro-Communications, Tokyo (JP)

(72) Inventors: Toru Shimuta, Nagaokakyo-shi (JP); Keiki Takadama, Tokyo (JP); Yuta Umenai, Tokyo (JP)

(21) Appl. No.: 16/737,018

(22) Filed: Jan. 8, 2020

Related U.S. Application Data
(63) Continuation of application No. PCT/JP2018/011287, filed on Mar. 22, 2018.

(30) Foreign Application Priority Data
Jul. 13, 2017 (JP) 2017-137372

(51) Int. Cl. A61B 5/00; A61B 5/0205; G16H 20/30; G16H 40/67

(52) U.S. Cl. CPC A61B 5/4857; A61B 5/4815; A61B 5/4035; A61B 5/486; A61B 5/024; A61B 5/02055; G16H 20/30; G16H 40/67; A61B 5/742

(57) ABSTRACT

A fatigue recovery support apparatus includes a circadian rhythm acquisition module which acquires a circadian rhythm, a pattern determination module which determines the acquired pattern of the circadian rhythm, a sleep determination module which determines a sleep quality, a relational data store which in advance stores relational data presenting a relationship among the circadian rhythm pattern, the sleep quality, and a fatigue recovery effect of sleep, and a recovery effect determination module which estimates the fatigue recovery effect of sleep based on the circadian rhythm pattern, the sleep quality, and the relational data.

### CROSS REFERENCE TO RELATED APPLICATIONS

The present application is a continuation of International application No. PCT/JP2018/011287, filed Mar. 22, 2018, which claims priority to Japanese Patent Application No. 2017-137372, filed Jul. 13, 2017, the entire contents of each of which are incorporated herein by reference.

### TECHNICAL FIELD

The present invention relates to a fatigue recovery support apparatus supporting recovery from fatigue.

### BACKGROUND OF THE INVENTION

In recent years, techniques of detecting human fatigue have been proposed. For example, Japanese Laid-Open Patent Publication No. 2010-201113 (Patent Document 1) discloses a fatigue-degree determination processing system which establishes a fatigue-degree determination reference value data for LF/HF values and compares the subject's LF/HF values calculated from pulse intervals (or heartbeat intervals) with the fatigue-degree determination reference value data to determine how fatigued the subject is (a fatigue degree).

In the fatigue-degree determination processing system described in Patent Document 1, for example, a-a intervals of acceleration pulse waves are separated into a low frequency component (LF: about 0.04 to 0.15 Hz) and a high frequency component (HF: about 0.15 to 0.40 Hz) by using a maximum entropy method (MEM), and the LF value is defined as a working value of the sympathetic nerve of the subject, while the HF value is defined as a working value of the parasympathetic nerve of the subject. This fatigue-degree determination processing system can evaluate sympathetic hyperactivity, and can evaluate a fatigue degree, by using the LF/HF values.

According to the fatigue-degree determination processing system described in Patent Document 1, a fatigue degree (level of fatigue) can be determined. However, no consideration is given to recovery from fatigue. Therefore, although a level of fatigue can be known, information that can be used as a guide for the subject's recovery from fatigue (reference information for fatigue recovery) cannot be obtained from the disclosed fatigue-degree determination processing system. Thus, the fatigue-degree determination processing system described in Patent Document 1 cannot contribute to the recovery of fatigue.

An object thereof is to provide a fatigue recovery support apparatus contributable to subject's recovery from fatigue.

### BRIEF DESCRIPTION OF THE INVENTION

A fatigue recovery support apparatus according to the present invention comprises: circadian rhythm acquisition module acquiring a circadian rhythm; pattern determination module determining a pattern of the circadian rhythm acquired by the circadian rhythm acquisition module; sleep determination module determining a sleep quality; relational data storage storing in advance relational data presenting a relationship among the circadian rhythm pattern, the sleep quality, and a fatigue recovery effect of sleep; and a recovery effect determination module estimating the fatigue recovery effect of sleep based on the circadian rhythm pattern, the sleep quality and the relational data.

According to the fatigue recovery support apparatus of the present invention, the relational data presenting the relationship among the circadian rhythm pattern, the sleep quality, and the fatigue recovery effect of sleep is stored in advance, and the fatigue recovery effect of sleep is estimated based on the acquired circadian rhythm pattern, the sleep quality, and the relational data stored in advance. Specifically, the subject's circadian rhythm and sleep quality are acquired and compared with a database (relational data/correlational data) obtained from a large number of people, for example, and the fatigue recovery effect of the subject's sleep can thereby be estimated. Therefore, for example, the subject can obtain information such as whether the circadian rhythm and the sleep (life rhythm) are matched or how the matching can preferably be achieved in the case of mismatching. As a result, a contribution can be made to the subject's recovery from fatigue.

The fatigue recovery support apparatus according to the present invention preferably includes: autonomic nerve activity measurement sensor measuring an autonomic nerve activity index; recovery degree determination module estimating a fatigue recovery degree based on a change in the autonomic nerve activity index measured by the measurement sensor; behavior storage module storing behavior information on behavior; and contribution degree determination module estimating a level of contribution of sleep and behavior to the fatigue recovery degree based on the fatigue recovery degree estimated by the recovery degree determination module, the behavior information stored in the behavior storage module, and the fatigue recovery effect of sleep estimated by the recovery effect determination module.

In this case, the level of contribution of sleep and behavior to the fatigue recovery degree is estimated based on the estimated fatigue recovery degree, the stored behavior information, and the estimated fatigue recovery effect of sleep. Specifically, a fatigue degree is actually measured, an estimated value is compared with an actual measurement value to obtain how much effect (influence) the behavior has, and respective levels of contribution of sleep and behavior to fatigue recovery can thereby be estimated. Therefore, the sleep condition and behavior contributable (having a high degree of contribution) to fatigue recovery can be estimated.

The fatigue recovery support apparatus according to the present invention preferably includes display presenting a sleep condition and/or a behavior suitable for fatigue recovery based on the level of contribution of sleep and behavior to the fatigue recovery degree estimated by the contribution degree determination module.

As a result, the sleep condition and the behavior contributable (having a high degree of contribution) to fatigue recovery can be presented to a subject.

In the fatigue recovery support apparatus according to the present invention, preferably, the autonomic nerve activity measurement sensor detects a heart rate or a pulse rate to measure an autonomic nerve activity index indicated by any of LF/HF, LF, HF, TP, and ccvTP.

In this case, by detecting a heart rate or a pulse rate that is relatively easy to detect, the autonomic nerve activity index can be measured that is indicated by any of LF/HF (low frequency component/high frequency component ratio), LF (low frequency component), HF (high frequency component), TP (total power (autonomic nerve activity amount)=LF+HF), and ccvTP (a value obtained by correcting TP with a heart rate during a measurement time).

In the fatigue recovery support apparatus according to the present invention, preferably, the circadian rhythm acquisition module measures at least one piece of biological data among body temperature, heart rate, pulse rate, and autonomic nerve activity index, and the pattern determination module determines the circadian rhythm pattern based on daily variation of the biological data measured by the circadian rhythm acquisition module.

In this case, the circadian rhythm pattern is determined from the daily variation of at least one piece of biological data among body temperature, heart rate, pulse rate, and autonomic nerve activity index. Therefore, the circadian rhythm pattern can be determined by measuring at least one piece of biological data among body temperature, heart rate, pulse rate, and autonomic nerve activity index.

In the fatigue recovery support apparatus according to the present invention, preferably, the sleep determination module measures at least any one piece of biological data among body motion, body temperature, heart rate, pulse rate, autonomic nerve activity index, respiratory rate, and brain wave during sleep to obtain at least any one piece of sleep data among sleep depth, duration of the each sleep depth, period, sleep time, bedtime, wake-up time, and a proportion of time of shallow sleep to total sleep time based on the biological data, and to determine the sleep quality based on the sleep data.

In this case, at least any one piece of biological data is measured among body motion, body temperature, heart rate, pulse rate, autonomic nerve activity index, respiratory rate, and brain wave during sleep to obtain at least any one piece of sleep data among sleep depth, duration of the each sleep depth, period, sleep time, bedtime, wake-up time, and a proportion of time of shallow sleep to total sleep time based on the biological data, and the sleep quality is determined based on the sleep data. Therefore, the sleep quality can be determined based on quantitative data (sleep data).

In the fatigue recovery support apparatus according to the present invention, preferably, the circadian rhythm acquisition module and the autonomic nerve activity measurement sensor are attached to a portable housing and detects a heart rate or a pulse rate to measure an autonomic nerve activity index indicated by any of LF/HF, LF, HF, TP, and ccvTP.

In this case, the autonomic nerve activity index indicated by any of LF/HF, LF, HF, TP, and ccvTP can be measured by detecting a heart rate or a pulse rate. Therefore, the circadian rhythm and the autonomic nerve activity index can be acquired by a portable device.

In the fatigue recovery support apparatus according to the present invention, preferably, the circadian rhythm acquisition module, the sleep determination module, and the autonomic nerve activity measurement sensor are attached to a housing wearable on a subject's body and detect a heart rate or a pulse rate to measure the autonomic nerve activity index indicated by any of LF/HF, LF, HF, TP, and ccvTP when accepting a subject's start operation or when automatically determining that a predetermined measurement start condition is satisfied.

In this case, the autonomic nerve activity index indicated by any of LF/HF, LF, HF, TP, and ccvTP can be measured by detecting a heart rate or a pulse rate. This enables a wearable device to acquire the circadian rhythm and determine the sleep quality. Additionally, a timing suitable for measurement can be determined to automatically perform the measurement.

In the fatigue recovery support apparatus according to the present invention, preferably, the circadian rhythm acquisition module, the sleep determination module, and the autonomic nerve activity measurement sensor are attached to portable housing or a housing wearable on a subject's body and detect a heart rate or a pulse rate to measure the autonomic nerve activity index indicated by any of TP, and ccvTP, and the sleep determination module determines a daily variation pattern of heart rate or pulse rate, and a daily variation pattern of TP or ccvTP to determine the sleep quality based on a correlation degree between the daily variation pattern of heart rate or pulse rate and the daily variation pattern of TP or ccvTP.

The inventor obtained knowledge that the correlation degree between the daily variation pattern of heart rate or pulse rate and the daily variation pattern of TP or ccvTP is correlated with the sleep quality. Therefore, according to the fatigue recovery support apparatus of the present invention, the daily variation pattern of heart rate or pulse rate is determined, the daily variation pattern of TP or ccvTP is determined, and the sleep quality is determined based on the correlation degree between the daily variation pattern of heart rate or pulse rate and the daily variation pattern of TP or ccvTP. Thus, the sleep quality can be determined based on quantitative data.

In the fatigue recovery support apparatus according to the present invention, preferably, the behavior storage module has input module accepting comments from subjects.

As a result, the subject's behavior information can be stored together with comments, and therefore, a behavior highly effective for fatigue recovery can more accurately be estimated.

The fatigue recovery support apparatus according to the present invention preferably further includes learning module learning the subject's circadian rhythm pattern, sleep quality, and fatigue recovery degree or subject's comments acquired in the past and reflecting a result of the learning on the relational data of the circadian rhythm pattern, the sleep quality, and the fatigue recovery effect of sleep.

In this case, the subject's circadian rhythm pattern, sleep quality, and fatigue recovery degree or subject's comments acquired in the past are learned, and the result of the learning is reflected on the relational data of the circadian rhythm classification, the sleep quality, and the fatigue recovery effect of sleep. Therefore, individual differences of subjects can be corrected by the learning, and the fatigue recovery support can be provided in accordance with the subjects.

According to this invention, a contribution can be made to subject's recovery from fatigue.

### BRIEF DESCRIPTION OF THE DRAWINGS

FIG. 1 is a block diagram showing a configuration of a fatigue recovery support apparatus according to a first embodiment.

FIG. 2 is a diagram showing an example (of a pattern) of circadian rhythm based on a body temperature change.

FIG. 3 is a diagram showing a relationship (correlation) between sleep and a fatigue recovery effect.

FIG. 4 is a block diagram showing a configuration of a fatigue recovery support apparatus according to a second embodiment.

FIG. 5 is a diagram showing an example of a grip-type measurement device.

FIG. 6 is a flowchart showing process procedures of a fatigue recovery recommendation process by the fatigue recovery support apparatus according to a second embodiment.

FIG. 7 is a block diagram showing a configuration of a fatigue recovery support apparatus (measurement device) according to a modification.

FIG. 8 is a block diagram showing a configuration of a fatigue recovery support apparatus according to a third embodiment.

FIG. 9 is a diagram showing an example of a neck-worn type measurement device.

FIG. 10 is a diagram showing an example of a wristwatch type measurement device.

FIG. 11 is a diagram showing an example of a chest-attached (worn) type measurement device.

FIG. 12 is a flowchart showing process procedures of an automatic measurement process by the fatigue recovery support apparatus according to the third embodiment.

FIG. 13 is a block diagram showing a configuration of a fatigue recovery support apparatus according to a fourth embodiment.

FIG. 14 is a diagram showing an example of respective daily variation patterns (correlation degree/inverse correlation degree) of body temperature and ccvTP, and an example of respective daily variation patterns (correlation degree/inverse correlation degree) of heart rate (HB) and ccvTP.

### DESCRIPTION OF THE PREFERRED EMBODIMENTS

Preferred embodiments of the present invention will now be described in detail with reference to the drawings. In the figures, the same reference numerals are used for the same or corresponding portions. Elements denoted by the same reference numerals in the various disclosed embodiments will not, as a generally rule, be repeatedly be described.

### First Embodiment

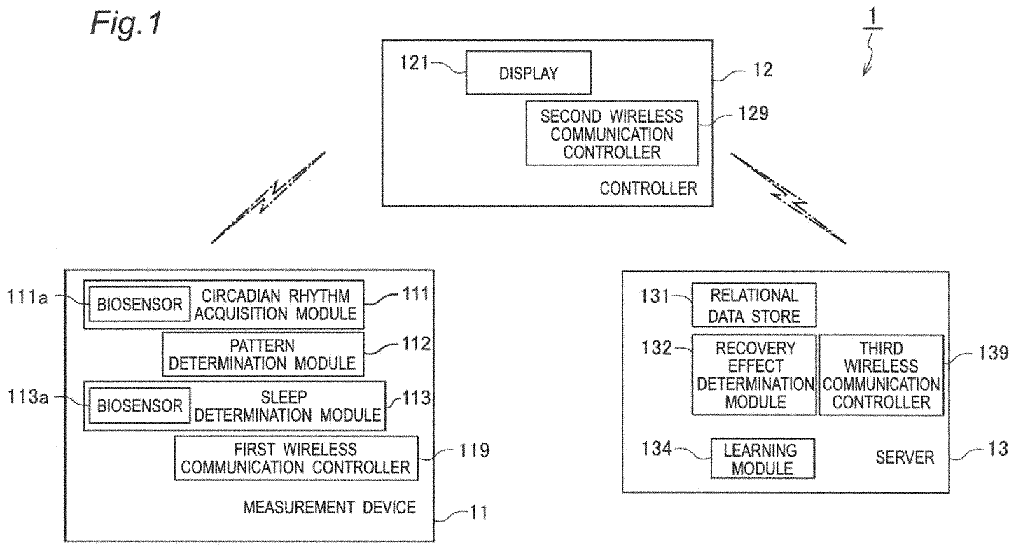

First, a configuration of a fatigue recovery support apparatus 1 according to a first embodiment will be described with reference to FIG. 1. FIG. 1 is a block diagram showing the configuration of the fatigue recovery support apparatus 1.

The fatigue recovery support apparatus 1 is an apparatus (system) which estimates a fatigue recovery effect on the subject during his or her sleep. The fatigue recovery support apparatus 1 primarily includes a measurement device 11, a controller 12, and a server 13 which are, in the present embodiment, communicably connected through wireless communication. As used throughout the specification, the term “module” refers to a programmed processor and/or equivalent hardware such as a programmed array logic. A single processor (and/or equivalent hardware) can support a plurality of modules which carry out one of more of the functions noted herein. Programs carrying out a specified function can run on the same or different processors. A given program can be run on more than one processor.

The measurement device 11 mainly includes a circadian rhythm acquisition module 111, a pattern determination module 112, sleep determination module 113, and a first wireless communication controller 119.

The circadian rhythm acquisition module 111 acquires a circadian rhythm of the subject. The circadian rhythm acquisition module 111 has a biosensor 11 la which measures at least anyone piece of biological data among body temperature, heart rate, pulse rate, and autonomic nerve activity index LF/HF (low frequency component/high frequency component ratio), LF (low frequency component), HF (high frequency component), TP (total power (autonomic nerve activity amount=LF+HF), ccvTP (a value obtained by correcting TP with a heart rate during a measurement time). In the present embodiment, a body temperature sensor (e.g., a chip thermistor or a resistance temperature detector) is used as the biosensor 111a, acquires body temperature data (biological data) at a series of time intervals and stores that data in a memory (for example, an SRAM and EEPROM).

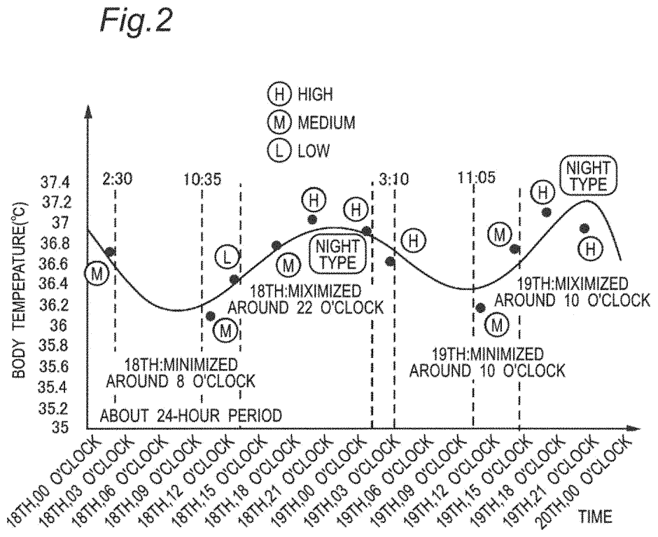

The pattern determination module 112 determines a circadian rhythm pattern based on daily variations of the acquired body temperature data (biological data). More specifically, the pattern determination module 112 first determines a circadian rhythm pattern through curve approximation of body temperature data having variations based on a preset approximation rule. An example of a circadian rhythm pattern based on a body temperature change is shown in FIG. 2. The vertical axis of FIG. 2 indicates body temperature (° C.), and the horizontal axis indicates date and time. A circular plot shown in FIG. 2 is body temperature data (body temperature measurement value), and an approximate curve is a circadian rhythm pattern. Multiple pattern types may be included (e.g., polynomial approximation, moving average, combination of multiple functions), and the pattern may be determined from an approximate correlation coefficient or a circadian rhythm pattern to the previous day.

Returning to FIG. 1, the pattern determination module 112 acquires a period and a peak time from the determined circadian rhythm pattern. The period and the peak time may be shifted (different) depending on a pattern type used. Furthermore, the pattern determination module 112 classifies the circadian rhythm in accordance with the acquired period, peak time, and pattern type. The circadian rhythm pattern can be determined and classified in the same manner even when the biological data other than body temperature is used.

More specifically, regarding the classification of circadian rhythm, for example, the classification can be made based on a difference in maximum and minimum of body temperature into a morning type (body temperature maximum: around 16 o'clock, minimum: around 4 o'clock), a night type (maximum: around 22 o'clock, minimum: around 10 o'clock), an inverted morning type (maximum: around 4 o'clock, minimum: around 16 o'clock), an inverted night type (maximum: around 10 o'clock, minimum: around 10 o'clock), etc. (see FIG. 2). Additionally, the classification can be made based on a difference in period (minimum peak interval) into a constant type (about 24 hours), a short period type (about 20 hours), a long period type (about 28 hours), an unknown type (a clear period is indeterminable) (See FIG. 2). According to this classification technique, the example shown in FIG. 2 is classified into the night type/constant type. The values of these peak times and period lengths are examples, and actual numerical values may be different. Additionally, when the circadian rhythm is disturbed, an amplitude of the pattern tends to decrease, and therefore, the amplitude of the pattern may be used as a criterion. Furthermore, a pattern determination accuracy tends to decrease when acquired data is insufficient in the circadian rhythm pattern determination method described above, and therefore, for example, a daily variation range, standard deviation, dispersion, etc. of the acquired body temperature data (biological data) may be substituted for the amplitude of the pattern to make the circadian rhythm pattern determination.

The sleep determination module 113 determines the quality of the subject's sleep. In the present embodiment, the sleep determination module 113 includes a biosensor 113a measuring at least any one piece of biological data among body motion, body temperature, heart rate, pulse rate, autonomic nerve activity index (LF/HF, LF, HF, TP, ccvTP), respiratory rate, and brain wave during sleep. In the present embodiment, a body motion sensor is used as the biosensor 113a. The body motion sensor can be, for example, a stationary body motion (vibration) sensor such as a sheet type sensor, a mat type sensor, or a sensor disposed next to the pillow, or a wearable type acceleration sensor. For example, when a seat type body motion sensor is used, the body motion data is acquired through the body motion sensor disposed under a mattress and transmitting the body motion data wirelessly (or by wire). The acquired body motion data is stored in time series in a memory such as SRAM and EEPROM, for example.

Based on the acquired body motion data (biological data), the sleep determination module 113 obtains at least any one piece of sleep data among sleep depth, duration of the each sleep depth, period, sleep time, bedtime, wake-up time, and a proportion of time of shallow sleep to total sleep time, and determines the sleep quality based on the sleep data. Therefore, the quality of the subject's sleep (i.e., the sleep quality) can be determined from bedtime, wake-up time, sleep time, time or proportion of shallow sleep(e.g., awakening, REM sleep, non-REM sleep stage 1), REM sleep period, etc. Since the heart rate and the respiratory rate can be estimated depending on a body motion data analysis method, the sleep quality analysis may be performed by using the heart rate and the respiratory rate in addition to the body motion. The sleep quality can be determined in the same manner even when the biological data other than body motion is used.

The acquired circadian rhythm classification/pattern, sleep quality, etc. are transmitted from the first wireless communication controller 119 via the controller 12 to the server 13. The first wireless communication controller 119 has a transmission function and a reception function, preferably based on BLE (Bluetooth (registered trademark) Low Energy), for example.

The controller 12 mainly includes a display 121 (which can be a visual and/or verbal device) and a second wireless communication controller 129. The display 121 can be, for example, an LCD display. The second wireless communication controller 129 has a transmission function and a reception function based on BLE, for example.

The controller 12 receives a fatigue recovery effect estimated value (described in detail below) transmitted from the server 13 using the second wireless communication controller 129, converts the value into an index presenting an effect of subject' s sleep on fatigue recovery, and displays the index on the display 121. The controller 12 analyzes the daily sleep quality and a transition of the fatigue recovery effect of sleep and displays directionality of sleep improvement (e.g., making bedtime earlier) on the display 121. Furthermore, the controller 12 has a function of instructing the measurement device 11 to start/stop measurement of circadian rhythm etc.

The server 13 mainly includes a relational data storage 131, a recovery effect determination module 132, a learning module 134, and a third wireless communication controller (wireless communication controller) 139.

The relational data storage 131 stores in advance relational data presenting a relationship (correlation) among the circadian rhythm classification, the sleep quality, and the fatigue recovery effect of sleep. The relational data may be, for example, data obtained by converting the correlation acquired among a large number of people into a function in advance.

The recovery effect determination module 132 estimates the fatigue recovery effect of sleep based on the circadian rhythm classification and the sleep quality received from the measurement device 11 as well as the relational data stored in the relational data storage 131. For example, if the circadian rhythm classification is the morning type/constant type described above, the sleep time and the proportion of shallow sleep tend to have a strong correlation with the fatigue recovery effect. On the other hand, in the case of the night type/constant type, the correlation between the sleep time and the fatigue recovery effect is weak, and the correlation with the bedtime (the time the subject goes to bed) tends to be strong.

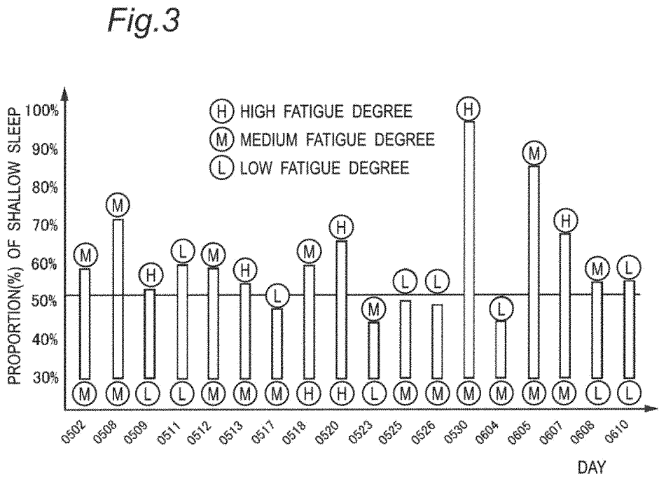

FIG. 3 shows a relationship (correlation) between sleep and the fatigue recovery effect. More particularly, FIG. 3 shows an example when the circadian rhythm classification is the morning type/constant type described above. Additionally, FIG. 3 shows a relationship between a proportion of shallow sleep to entire sleep (bar graph) and a fatigue degree estimated from the autonomic nerve index before and after sleep (three levels: low<medium<high). As shown in FIG. 3, when the proportion of shallow sleep is larger, the fatigue degree of the next day tends to be higher (“medium” and “high” described on the upper side of the bar graph tend to increase).

Therefore, as described above, data (relational data) obtained by converting such correlation acquired among a large number of people into a function in advance is stored in the relational data storage 131. The recovery effect determination module 132 receives the circadian rhythm classification and the sleep quality of a subject, refers to the relational data, obtains an estimated value of the fatigue recovery effect of sleep, and outputs the result (the estimated value of the fatigue recovery effect of sleep). The acquired estimated value of the fatigue recovery effect of sleep is transmitted via the third wireless communication controller 139 to the controller 12.

Although the relationship among the circadian rhythm classification, the sleep quality, and the fatigue recovery effect of sleep is set in advance by using a measurement result of a large number of people as a default, a large deviation may occur due to an individual difference depending on the individual subject using the fatigue recovery apparatus 1. Therefore, the learning module 134 (in this embodiment) estimates an individual difference based on a subject's subjective degree of recovery from fatigue feeling to correct the relational data. Although the fatigue feeling in this case is different from objective fatigue, the learning module 134 corrects the relationship among the circadian rhythm classification, the sleep quality, and the fatigue recovery effect of sleep such that variation among a plural of days of the fatigue feeling becomes closer to the variation among a plural of days of the fatigue recovery effect of sleep.

The third wireless communication controller 139 has a transmission function and a reception function based on BLE, for example. As described above, the third wireless communication controller 139 transmits the estimated value of the fatigue recovery effect of sleep to the controller 12.

As described above, according to this embodiment, the relational data presenting the relationship among the circadian rhythm classification, the sleep quality, and the fatigue recovery effect of sleep is stored in advance, and the fatigue recovery effect of sleep is estimated based on the acquired circadian rhythm pattern, the sleep quality, and the relational data stored in advance. Specifically, the subject's circadian rhythm and sleep quality are acquired and compared with a database (relational data/correlational data) obtained from a large number of people, for example, and the fatigue recovery effect of the subject's sleep can thereby be estimated. Therefore, for example, the subject can obtain information such as whether the circadian rhythm and the sleep (life rhythm) are matched or how the matching can preferably be achieved in the case of mismatching. As a result, a contribution can be made to the subject's recovery from fatigue.

According to this embodiment, the circadian rhythm pattern is determined from the daily variation of the body temperature data. Therefore, the circadian rhythm pattern can be determined by measuring the body temperature data.

According to this embodiment, the body motion data during sleep is measured, and at least any one piece of sleep data is obtained, based on the body motion data, out of sleep depth, duration of the each sleep depth, period, sleep time, bedtime, wake-up time, and a proportion of time of shallow sleep to total sleep time, and the sleep quality is determined based on the sleep data. Therefore, the sleep quality can be determined based on quantitative data (sleep data).

According to this embodiment, the subject's circadian rhythm classification and sleep quality acquired in the past are learned, and a result of the learning is reflected on the relational data of the circadian rhythm classification, the sleep quality, and the fatigue recovery effect of sleep. Therefore, individual differences of subjects can be corrected by the learning, and the fatigue recovery support can be provided in accordance with the subjects.

### Second Embodiment

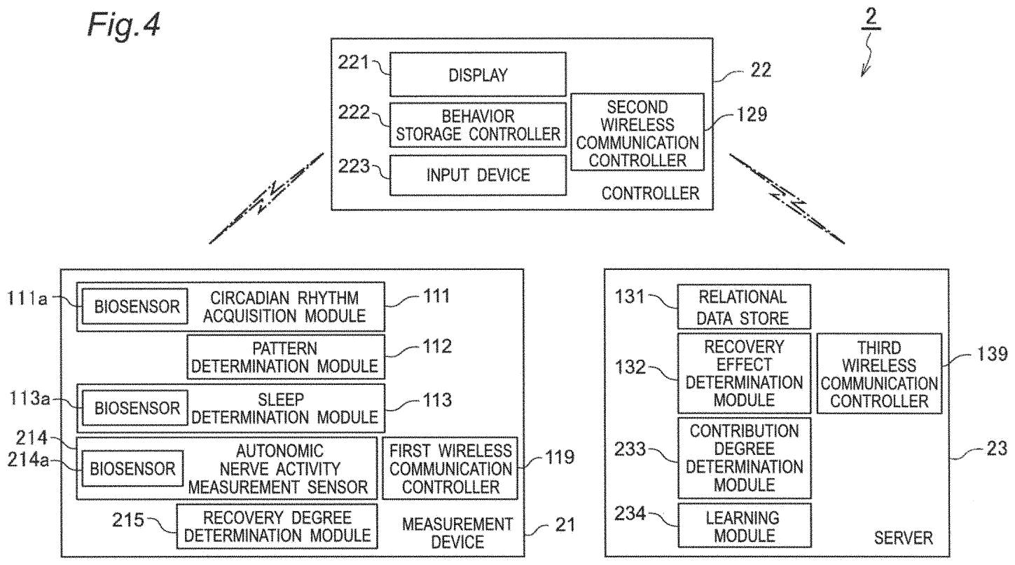

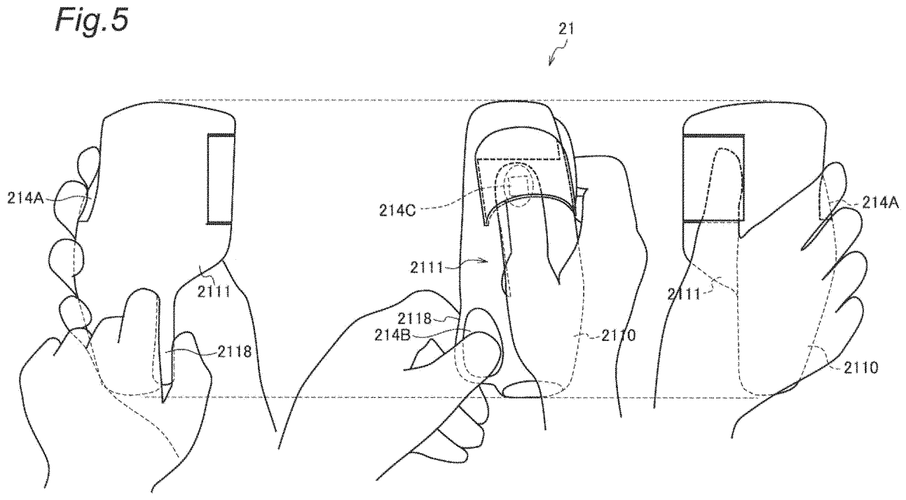

A fatigue recovery support apparatus 2 according to a second embodiment will be described with reference to FIGS. 4 and 5. The configurations which are the same as or similar to the first embodiment will be described in simplified manner or will not be described at all. The differences between the two embodiments will mainly be described. FIG. 4 is a block diagram showing a configuration of the fatigue recovery support apparatus 2. FIG. 5 is a diagram showing an example of a grip-type measurement device 21. In FIGS. 4 and 5, the same or equivalent constituent elements as the first embodiment are denoted by the same reference numerals.

The fatigue recovery support apparatus 2 estimates sleep condition and behavior contributable (having a high degree of contribution) to fatigue recovery to assist subject's recovery from fatigue. The fatigue recovery support apparatus 2 is different from the fatigue recovery support apparatus 1 described above in that the device includes a measurement device 21, a controller 22, and a server 23 instead of the measurement device 11, the controller 12, and the server 13. The measurement device 21 is different from the measurement device 11 described above in that the device further includes an autonomic nerve activity measurement sensor 214 and a recovery degree determination module 21.

Similarly, the controller 22 is different from the controller 12 described above in that the device further includes a behavior storage controller 222 and an input device 223 and that the device includes a display 221 instead of the display 121.

The server 23 is different from the server 13 described above in that the server further includes a contribution degree determination module 233 and that the server includes a learning module 234 instead of the learning module 134. The other configurations are the same as or similar to the first embodiment described above and therefore will not be described in detailed.

The autonomic nerve activity measurement sensor 214 measures an autonomic nerve activity index. More specifically, the autonomic nerve activity measurement sensor 214 has a biosensor 214a detecting a heart rate (or pulse rate) and detects a heart rate (or pulse rate) to measure the autonomic nerve activity index indicated by any of LF/HF, LF, HF, TP, and ccvTP.

The autonomic nerve activity index can be obtained from variation in the measured heart rate or pulse rate. More specifically, the autonomic nerve activity index can be calculated by a frequency analysis from heartbeat intervals or pulse intervals, for example. Specifically, the frequency analysis (spectrum analysis) of heartbeat variations (variations in R-R intervals) can be conducted by using a technique such as fast Fourier transform to acquire a low frequency component (LF) up to 0.15 Hz mainly reflecting the sympathetic nerve function (partially including the parasympathetic nerve), a high frequency component (HF) of 0.15 Hz or higher reflecting the parasympathetic nerve function, and a ratio (LF/HF) of the low frequency component/the high frequency component. Alternatively, after calculating acceleration pulse waves through secondary differentiation of waveforms of brain waves and obtaining variations in a-a intervals (pulse intervals) corresponding to variations in R-R intervals of an electrocardiogram from the obtained waveforms of the acceleration pulse waves, the frequency analysis of the time variations in R-R intervals can be conducted to obtain the autonomic nerve activity index from the result thereof.

The autonomic nerve activity index is preferably acquired several times a day under fixed conditions. For measurement conditions, it is important to be in a resting state in a sitting position. Additionally, it is desirable to avoid a timing immediately after behavior affecting the autonomic nerve activity, such as exercising (including walking), taking a meal, and bathing, since the autonomic nerve activity index may be affected. The measured autonomic nerve activity index is output to the recovery degree determination module 215.

In this embodiment, the measurement device 21 is a grip type measurement device wherein the biosensor 214a preferably detects the heart rate or the pulse rate and is attached to a portable grip-type housing. FIG. 5 shows an example of the grip-type measurement device 21.

In this embodiment, the measurement device 21 is a grip-type measurement device capable of acquiring an electrocardiographic signal and a photoelectric pulse wave signal and measuring a heart rate, a pulse rate, a body temperature, etc. when gripped by a subject. The measurement device 21 has a main body part 2110 formed into a substantially spheroid shape gripped with the thumb and the other four fingers of one hand (e.g., the right hand) by a subject during measurement. Aside surface of the main body part 2110 includes a plate-shaped flange part 2118 disposed in a protruding manner in a direction substantially orthogonal to a protruding direction of a stopper part 2111 (i.e., in a lateral direction). The flange part 2118 is disposed to extend along the axial direction of the main body part 2110 (i.e., from the proximal end side to the distal end side).

A first electrocardiographic electrode 214A is arranged such that when the main body part 2110 is gripped with one hand (e.g., the right hand), a finger (e.g., the index finger and/or the middle finger) of the one hand comes into contact therewith. The first electrocardiographic electrode 214A may be disposed such that the thumb of one hand (e.g., the right hand) comes into contact therewith.

On the other hand, a front-side surface (and/or a back-side surface) of the flange part 2118 is provided with a second electrocardiographic electrode 214B formed into, for example, an elliptical shape for detecting an electrocardiographic signal. Specifically, the second electrocardiographic electrode 214B is arranged such that when the flange part 2118 is pinched (sandwiched) by fingers (e.g., the thumb and the index finger) of the other hand (e.g., the left hand), a finger (e.g., the thumb and/or the index finger) of the other hand comes into contact therewith. Therefore, when the subject grips the main body part 2110 and the flange part 2118 of the grip-type measurement device 21, the first electrocardiographic electrode 214A and the second electrocardiographic electrode 214B are brought into contact with the subject's left and right hands (fingertips) so that an electrocardiographic signal corresponding to a potential difference between the subject's left and right hands is acquired.

The main body part 2110 is provided with a photoelectric pulse wave sensor 214C. The photoelectric pulse wave sensor 214C has a light emitting element and a light receiving element and acquires the photoelectric pulse wave signal from the tip of the thumb restricted by the stopper part 2111. The photoelectric pulse wave sensor 214C is a sensor optically detecting a photoelectric pulse wave signal by using the light absorption characteristics of blood hemoglobin.

Returning to FIG. 4, the recovery degree determination module 215 of the measurement device 21 estimates a fatigue recovery degree based on the amount or rate of change of the autonomic nerve activity index measured by the autonomic nerve activity measurement sensor 214. Specifically, the recovery degree determination module 215 estimates fatigue from the measured autonomic nerve activity index and calculates the fatigue recovery degree from daily changes in fatigue. The measurement of the autonomic nerve activity index for calculating the fatigue recovery degree is desirably performed under the same conditions (such as measurement time and measurement location) every day. To suppress variations in measurement conditions, the measurement data acquired for several times maybe processed for an average or a median before use. The estimated fatigue recovery degree is transmitted by the first wireless communication controller 119 via the controller 22 to the server 23. The function of the recovery degree determination module 215 may be implemented on the server 23 side.

For example, the autonomic nerve activity index of a subject of the night type/long period type in the circadian rhythm classification was improved when the subject temporarily went to bed early and got up early to achieve a morning type circadian rhythm. More specifically, the LF/HF value decreased from 5.26 to 4.57, and the ccvTP value increased from 5.10 to 5.44 (a decrease in LF/HF and an increase in ccvTP both indicate a reduction in fatigue degree). Additionally, no clear correlation was observed in the same subject between the proportion of shallow sleep and the autonomic nerve activity index.

The behavior store 222 of the controller 22 stores information (behavior information) on subject's behavior. The behavior storage controller 222 has an input device 223 (e.g., a keyboard, a touch sensor or a speech recognition device) which allows the subject (or someone working with the subject) to enter comments. To simplify the input, it is important to prepare frequently-used comments in a selective manner so as to simplify subject's operations and reduce troubles. For example, the autonomic nerve activity and the body temperature are likely to be affected by walking, exercising, taking a meal, bathing, and sleeping, as well as by going out, working, feeling warm or cold, a state of mind, feeling of fatigue, a degree of mental fatigue, a degree of physical fatigue, and sleepiness. The comments input can be simplified by preparing selection buttons for frequently-used comments. Additionally, by further disposing a comment input module allowing free input, the input is enabled even in special situations. By inputting these contents before and after the measurement, the measurement conditions can be limited, and behaviors/conditions correlated with recovery from fatigue/increase in fatigue can be extracted by using these as the measurement conditions at the time of data analysis. For example, if a correlation exists between a comment “cold” and an increase in fatigue, an advice for improvement can be presented to prompt a subject to refrain from behavior associated with feeling “cold”. The acquired and stored behavior information (including comments) is transmitted via the second wireless communication controller 129 to the server 23.

In addition to the display content described above, the display 221 presents sleep condition and/or behavior suitable for fatigue recovery based on a level of contribution of sleep and behavior to the fatigue recovery degree (described in detail later) estimated by the contribution degree determination module 233 of the server 23.

The contribution degree determination module 233 of the server 23 estimates the level of contribution of sleep and behavior to the fatigue recovery degree based on the received fatigue recovery degree and behavior information as well as the fatigue recovery effect of sleep estimated by the recovery effect determination module 232. More specifically, to accurately calculate the fatigue recovery degree, the contribution degree determination module 233 compares the fatigue recovery degree with the fatigue recovery effect estimated value and thereby estimates the contribution of sleep and behavior to the fatigue recovery degree. For example, recommendation/improvement information is generated in accordance with the following criteria:

(1) In the case of “fatigue recovery effect of sleep >0”, the sleep is recommended.

(2) In the case of “fatigue recovery effect of sleep ≤0”, an improvement of sleep is recommended.

(3) In the case of “fatigue recovery degree>fatigue recovery effect of sleep”, it is determined that fatigue recovery is achieved by behavior, and the behavior on the day (the current day) is recommended. Additionally, based on learning of comments entered on days when fatigue recovery is achieved by previous behavior, relevant comments are extracted to narrow down behavior effective for fatigue recovery, and the behavior is recommended.

(4) In the case of “fatigue recovery degree<fatigue recovery effect of sleep”, it is determined that fatigue is an increased by behavior, and an improvement of the behavior on the day (the current day) is recommended. Additionally, based on learning of comments (and exercise intensity and exercise time) entered on days when fatigue is increased by previous behavior, relevant comments are extracted to narrow down behavior increasing fatigue, and an improvement of the behavior is recommended.

Since fatigue is accumulated, the fatigue unable to be removed by sleeping affects the fatigue degree of the next day. Therefore, the accuracy of estimation of the contribution of sleep and behavior to the fatigue recovery degree can be improved by using data of several days rather than data of a single day. The estimated level of contribution of sleep and behavior to the fatigue recovery degree is output by the third wireless communication controller 139 to the controller 22.

The learning module 234 learns the circadian rhythm classification, the sleep quality, and the fatigue recovery degree of the subject or the subject's input comments to the previous day so as to correct the relationship (correlation) among the circadian rhythm classification, the sleep quality, and the fatigue recovery effect of sleep stored in advance. For example, if the correlation is low between the fatigue recovery degree and the estimated value of the fatigue recovery effect of sleep, the learning module 234 corrects the relationship among the circadian rhythm classification, the sleep quality, and the fatigue recovery effect of sleep such that variation among a plural of days of the fatigue recovery degree obtained from actually measured fatigue measurement results becomes closer to the variation among a plural of days of the estimated value of the fatigue recovery effect of sleep.

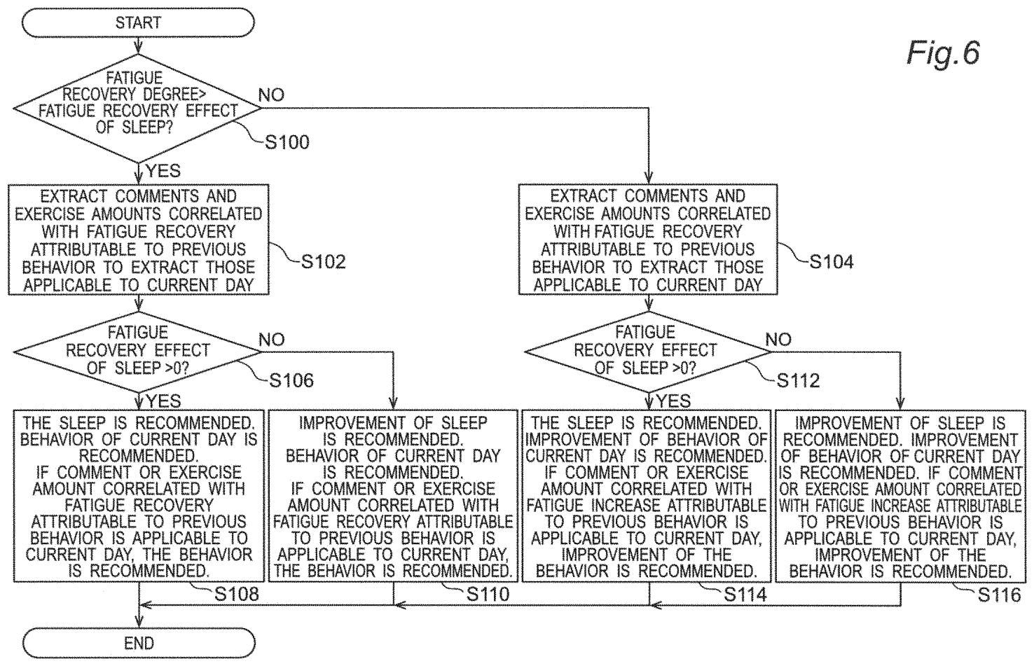

The operation of the fatigue recovery support apparatus 2 will be described with reference to FIG. 6. FIG. 6 is a flowchart showing process procedures of a fatigue recovery recommendation process by the fatigue recovery support apparatus 2.

At step S100, it is determined whether the fatigue recovery degree exceeds the fatigue recovery effect of sleep. If the fatigue recovery degree exceeds the fatigue recovery effect of sleep, the process goes to step S102. If the fatigue recovery degree does not exceed the fatigue recovery effect of sleep, the process goes to step S104.

At step S102, comments and exercise amounts correlated with fatigue recovery attributable to previous behavior are extracted to extract those applicable to the day on which the process is executed (hereinafter referred to as the current day).

Subsequently, at step S106, it is determined whether the fatigue recovery effect of sleep is positive (>0). If the fatigue recovery effect of sleep is positive (>0), the process goes to step S108. On the other hand, if the fatigue recovery effect of sleep is not positive (≤0), the process goes to step S110.

At step S108, the sleep is recommended, the behavior of the current day is recommended, and if comments or an exercise amount correlated with fatigue recovery attributable to a previous behavior is applicable to the current day, the behavior is recommended. Subsequently, the process is temporarily ended.

On the other hand, at step S110, an improvement of sleep is recommended, the behavior of the current day is recommended, and if comments or an exercise amount correlated with fatigue recovery attributable to a previous behavior is applicable to the current day, the behavior is recommended. Subsequently, the process is temporarily ended.

On the other hand, if the determination of step S100 is negative, at step S104, comments and exercise amounts correlated with fatigue recovery attributable to previous behavior are extracted to extract those applicable to the day on which the process is executed (the current day).

Subsequently, at step S112, it is determined whether the fatigue recovery effect of sleep is positive (>0). If the fatigue recovery effect of sleep is positive (>0), the process goes to step S114. On the other hand, if the fatigue recovery effect of sleep is not positive (≤0), the process goes to step S116.

At step S114, the sleep is recommended, an improvement of the behavior of the current day is recommended, and if comments or an exercise amount correlated with fatigue recovery attributable to a previous behavior is applicable to the current day, the behavior is recommended. Subsequently, the process is temporarily ended.

On the other hand, at step S116, an improvement of sleep is recommended, an improvement of the behavior of the day is recommended, and if comments or an exercise amount correlated with fatigue recovery attributable to a previous behavior is applicable to the day, the behavior is recommended. Subsequently, the process is temporarily ended.

According to this embodiment, the level of contribution of sleep and behavior to the fatigue recovery degree is estimated based on the fatigue recovery degree, the behavior information, and the fatigue recovery effect of sleep. Specifically, a fatigue degree is actually measured, an estimated value is compared with an actual measurement value to obtain how much effect (influence) the behavior has, and respective levels of contribution of sleep and behavior to fatigue recovery are estimated. Therefore, the sleep condition and behavior contributable (having a high degree of contribution) to fatigue recovery can be estimated.

According to this embodiment, a sleep condition and/or a behavior suitable for fatigue recovery are presented based on the level of contribution of sleep and behavior to the fatigue recovery degree. Therefore, the sleep condition and the behavior contributable (having a high degree of contribution) to fatigue recovery can be presented to the subject.

According to this embodiment, by detecting a heart rate or a pulse rate that is relatively easy to detect, the autonomic nerve activity index indicated by any of LF/HF, LF, HF, TP, and ccvTP can be measured. Therefore, the circadian rhythm and the autonomic nerve activity index can be acquired by a portable (graspable) device.

According to this embodiment, the subject's behavior information can be stored together with comments, and therefore, a behavior highly effective for fatigue recovery can more accurately be estimated.

### Modification

In the second embodiment described above, a body temperature sensor is used for measuring the circadian rhythm, and a heart rate sensor (or pulse rate sensor) is used for measuring the autonomic nerve activity index. However, to simplify the device configuration and make the operation easier, a circadian rhythm acquisition module 211 and an autonomic nerve activity measurement sensor 214 may use a common heart rate or pulse rate sensor 211a.

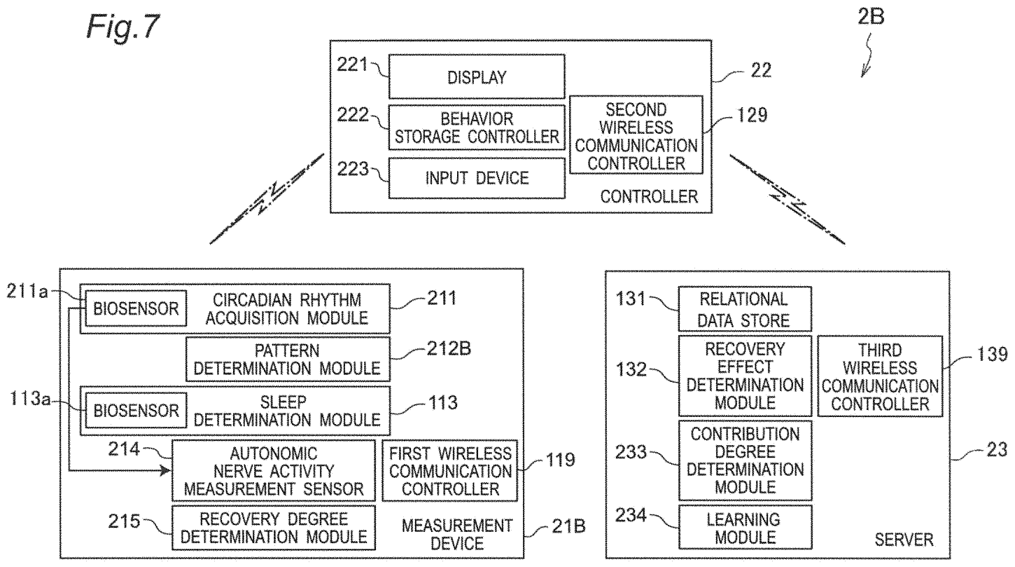

A fatigue recovery support apparatus 2B according to a modification of the second embodiment will be described with reference to FIG. 7. The configurations which are the same as or similar to the second embodiment will be described in simplified manner or will not be described at all. The differences between the two embodiments will mainly be described. FIG. 7 is a block diagram showing a configuration of the fatigue recovery support apparatus 2B. In FIG. 7, the same or equivalent constituent elements as the second embodiment are denoted by the same reference numerals.

In the fatigue recovery support apparatus 2B, the circadian rhythm measurement sensor 211 and the autonomic nerve activity measurement sensor 214 share a common heart rate or pulse rate sensor 211a. By way of example, an electrocardiographic sensor or a ballistocardiographic sensor can be used. A photoelectric pulse wave sensor, a piezoelectric pulse wave sensor, or an oxygen saturation sensor, by way of example, can be used as the pulse rate sensor.

When the autonomic nerve activity index acquired from the heart rate (or pulse rate) sensor is used for determining the circadian rhythm, a pattern determination module 212B organizes the heart rate (pulse rate) and the autonomic nerve activity index measured during wakefulness by the measurement time and obtains a period of change, times of maximum and minimum points, and an amplitude of change to estimate the circadian rhythm. To increase determination accuracy, the heart rate (or pulse rate) is measured about five times a day. The determination accuracy of the circadian rhythm can be improved by using the changes of the last several days instead of one day. If the subject is not in a resting state at the time of measurement, the heart rate (pulse rate) and the autonomic nerve activity index are affected, and the circadian rhythm determination accuracy is also reduced, and therefore, the device is preferably provided with a function of confirming whether the subject is in a resting state.

The pattern determination module 212B classifies the circadian rhythm after estimating the circadian rhythm. In this case, since an error of circadian rhythm estimation may increase due to an influence of walking, exercising, taking a meal, and bathing on the heart rate and the autonomic nerve activity index, excessively fine classification makes the influence of the error stronger and instead leads to a loss of correlation. Therefore, the appropriate number of classifications is about 4 to 8. The other configurations are the same as or similar to the second embodiment (the fatigue recovery support apparatus 2) described above and therefore will not be described in detailed.

According to this modification, since the circadian rhythm measurement sensor 211 and the autonomic nerve activity measurement sensor 214 use a common heart rate (or pulse rate) sensor 211a, the configuration can be simplified and the operation can be made easier.

### Third Embodiment

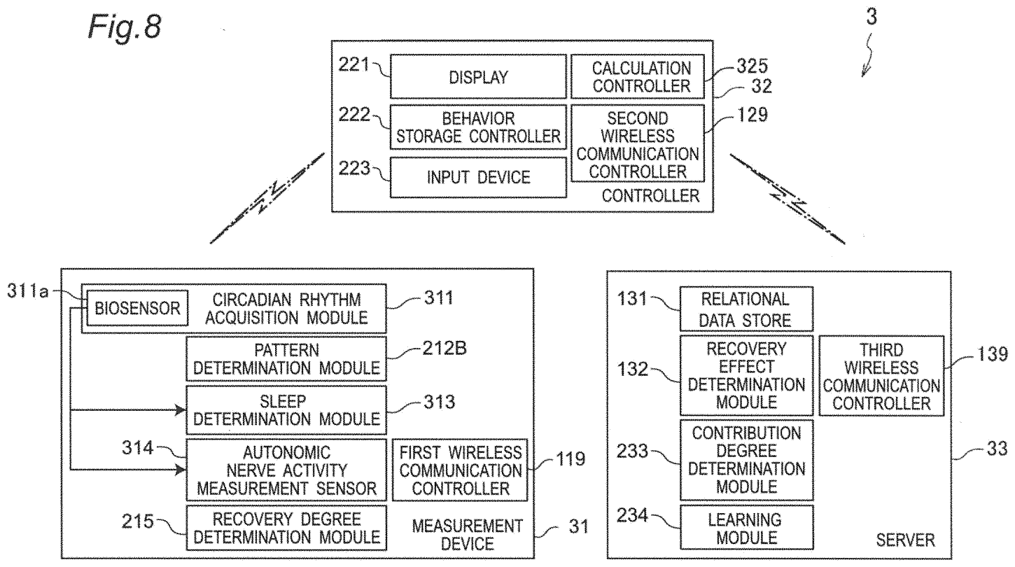

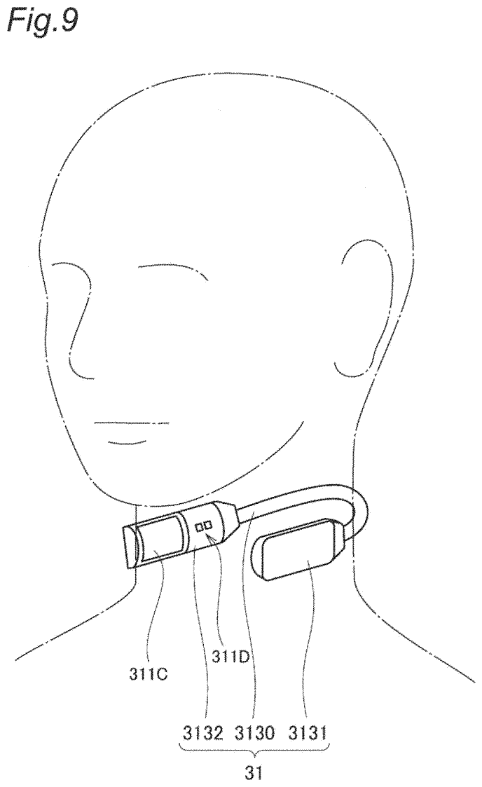

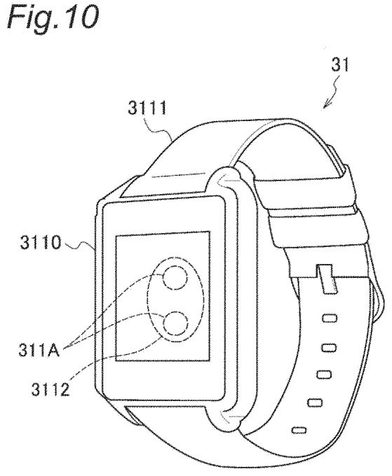

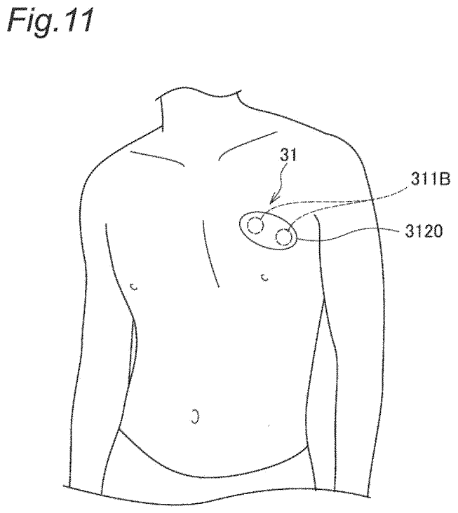

A fatigue recovery support apparatus 3 according to a third embodiment will be described with reference to FIGS. 8 to 11. The configurations same as or similar to the second embodiment will be described in simplified manner or will not be described, and differences will mainly be described. FIG. 8 is a block diagram showing a configuration of the fatigue recovery support apparatus 3. FIG. 9 is a diagram showing an example of a neck-worn type measurement device 31. FIG. 10 is a diagram showing an example of a wristwatch type measurement device 31. FIG. 11 is a diagram showing an example of a chest-worn (attached) type measurement device 31. In FIGS. 8 to 11, the same or equivalent constituent elements as the second embodiment (or the modification thereof) are denoted by the same reference numerals.

The fatigue recovery support apparatus 3 includes a wearable measurement device 31 and is provided with an automatic measurement function. The fatigue recovery support apparatus 3 is different from the fatigue recovery support apparatus 2 described above in that the device includes the measurement device 31 and a controller 32 instead of the measurement device 21 and the controller 22.

A circadian rhythm acquisition module 311, a sleep determination module 313, and an autonomic nerve activity measurement sensor 314 constituting the measurement device 31 share a common biosensor 313a (heart rate sensor or pulse rate sensor).

The measurement device 31 is attached to a housing which can be worn on the subject's body and detects a heart rate or a pulse rate to measure the autonomic nerve activity index indicated by any of LF/HF, LF, HF, TP, and ccvTP when accepting a subject's start operation or when automatically determining that a measurement start condition is satisfied.

For example, a neck-worn type worn on the neck, a wristwatch type worn on the wrist, or a chest-attached type attached to the chest is preferably used for the wearable measurement device 31.

The neck-worn type can have either a configuration in which the pulse rate is measured by a photoelectric pulse wave sensor or a configuration in which the heart rate is measured by an electrocardiographic sensor having multiple electrocardiographic electrodes. Although causing relatively significant discomfort during exercise, the neck-worn type does not cause such discomfort in daily life. The neck-worn type provides favorable measurement stability next to the chest-attached type and can sufficiently perform the autonomic nerve activity measurement. Since the body surface temperature in the vicinity of the carotid artery is close to the deep body temperature, the neck-worn type can estimate the deep body temperature and can estimate the circadian rhythm from the deep body temperature as in the chest-attached type.

FIG. 9 shows an example of the neck-worn type measurement device 31. The measurement device 31 includes a substantially U-shaped neckband 3130 elastically worn to sandwich the subject's neck from the back side of the neck, and a pair of sensor modules 3131, 3132 disposed at both ends of the neckband 3130 and thereby coming into contact with both sides of the subject's neck. The sensor module 3132 (3131) mainly has an electrocardiographic electrode (conductive cloth) 311C formed into a rectangular planar shape. The one sensor module 3132 includes a photoelectric pulse wave sensor 311D in addition to the configuration described above. The photoelectric pulse wave sensor 311D optically detects a photoelectric pulse wave signal by using the light absorption characteristics of blood hemoglobin.

On the other hand, the wristwatch type preferably has a configuration in which the pulse rate is measured by a photoelectric pulse wave sensor. The wristwatch type has an advantage of reducing subject's discomfort; however, the movement of the arm is larger than the other parts so that the measurement stability becomes relatively low, and the accuracy may become insufficient for the autonomic nerve activity measurement, which requires the measurement accuracy for fluctuation in heart rate for each heartbeat. Additionally, the movement is often not interlocked with the trunk of the body (e.g., in the case of waving the hand), and the determination accuracy for exercise intensity may be reduced.

FIG. 10 shows an example of the wristwatch-type measurement device 31. The wristwatch-type measurement device 31 includes a main body part 3110, a belt 3111 attached to the main body part 3110, and a pulse wave sensing module 3112 disposed on the back surface of the main body part 3110. A photoelectric pulse wave sensor 311A is disposed on the inner surface side of the pulse wave sensing module 3112. Therefore, when the subject wears this wristwatch-type measurement device 31 on the wrist of one hand (e.g., the left hand), the photoelectric pulse wave sensor 311A comes into contact with the wrist of the subject and performs the measurement of the pulse wave number etc.

The chest-attached type preferably has a configuration in which the heart rate is measured by an electrocardiographic sensor having multiple electrocardiographic electrodes. The chest-attached type causes relatively significant discomfort in the prone position. Additionally, if an adhesive tape is used for attachment to the chest, skin irritation may occur. Although having a disadvantage that the adhesive tape needs to be replaced, the chest-attached type provides the best measurement stability among the three types. Since the device is attached to the trunk of the body, the deep body temperature (core temperature) can be estimated from the heat flux due to the body surface temperature, and the circadian rhythm can be estimated from the deep body temperature. The device can be fixed to the chest by a belt instead of the adhesive tape. Although the adhesive tape may come off due to sweating, the device fixed by the belt does not come off due to sweating. However, tightening of the belt causes relatively significant discomfort.

FIG. 11 shows an example of the chest-attached (worn) type measurement device 31. The measurement device 31 includes a main body part 3120 that can be affixed to the chest of the subject, and two (or two or more) electrocardiographic electrodes (gel electrodes) 311B detachably attached to the main body part 3120. When an electrocardiographic signal etc. are measured by using the measurement device 31, the measurement device 31 is affixed to (worn on) the chest to bring the electrocardiographic electrodes (gel electrodes) 311B into contact with the chest. As a result, the electrocardiographic signal is detected by the electrocardiographic electrodes (gel electrodes) 311B.

Returning to FIG. 8, to record subject's behaviors, the controller 32 further includes, for example, an acceleration sensor (not shown) measuring acceleration, a GPS (not shown) acquiring a position, and a calculation controller 325 obtaining exercise intensity, movement history, etc. from output of these sensors. Among the behaviors, the exercise intensity and the movement history can automatically be acquired by using the acceleration sensor and the GPS, respectively, so that the trouble of comment input can be eliminated to prevent the subject from feeling bothersome.

The controller 32 has a start switch (not shown) for requesting start/stop of measurement. The measurement is preferably started after the subject enters the resting state. The measurement is started by the subject pressing the start switch.

Alternatively, instead of being started by the subject, the measurement may automatically be performed based on determination made on whether the subject is in the rest state in accordance with acceleration data. The controller 32 determines that the subject is in the resting state when the acceleration is not significantly changed continuously for a predetermined time and sends an instruction to the measurement device 31 for starting the measurement. The controller 32 also determines whether a large body motion has occurred during the measurement and outputs an alert if the large body motion has occurred. Furthermore, if the accuracy of calculation of the autonomic nerve activity index is possibly significantly reduced, the controller 32 instructs the measurement device 31 to perform the measurement again (remeasurement).

The device may constantly measure acceleration to calculate exercise intensity and determine from the exercise intensity whether the subject is walking, exercising, or resting before and after the measurement so as to determine reliability of an analysis result (e.g., exercise immediately before measurement results in determination of low reliability). Since it takes time to reach the resting state after exercising or walking, the measurement may not be started for a predetermined time. Furthermore, the device may be configured to constantly measure the heart rate/pulse rate to extract a time zone in which the resting state continues for the time required for the analysis during or after the measurement and to conduct analysis by using the data of the time zone. The other configurations are the same as or similar to the fatigue recovery support apparatus 2 (or the modification thereof) described above and therefore will not be described in detailed.

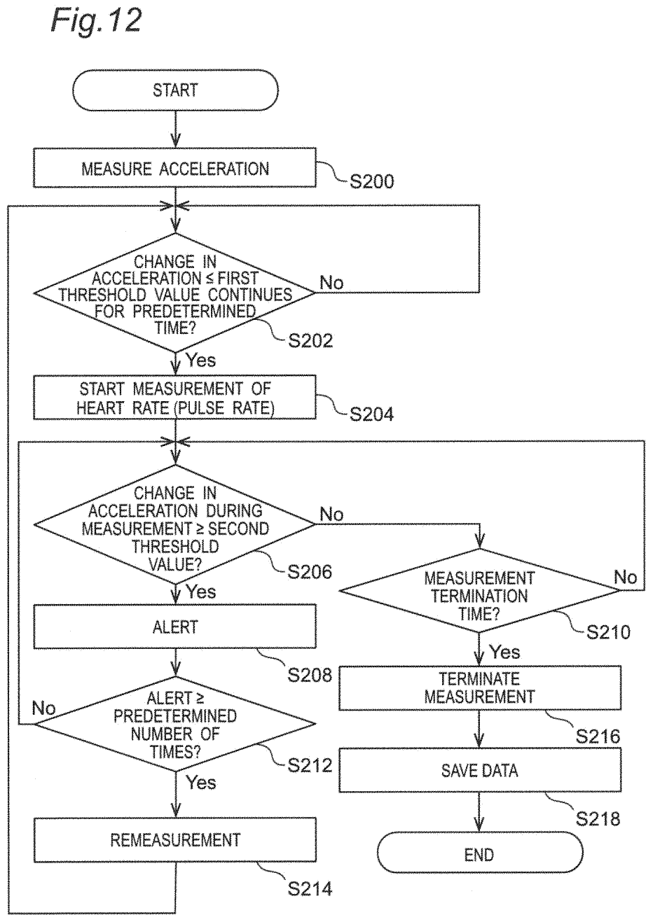

The operation of the fatigue recovery support apparatus 3 will be described with reference to FIG. 12. FIG. 12 is a flowchart showing process procedures of an automatic measurement process by the fatigue recovery support apparatus 3.

First, at step S200, acceleration is detected and read. Subsequently, at step S202, it is determined whether a change in acceleration is continuously equal to or less than a first threshold value for a predetermined time or more. If a change in acceleration is continuously equal to or less than the first threshold value for a predetermined time or more, the process goes to step S204. On the other hand, if a change in acceleration is not continuously equal to or less than the first threshold value for a predetermined time or more, this process is repeatedly executed until the condition is satisfied.

At step S204, measurement of the heart rate (or pulse rate) is started. Subsequently, at step S206, it is determined whether the change in acceleration during measurement is equal to or greater than a second threshold value. If the change in acceleration during measurement is equal to or greater than the second threshold value, the process goes to step S208. On the other hand, if the change in acceleration during measurement is less than the second threshold value, the process goes to step S210.

At step S208, an alert is output. Subsequently, at step S212, it is determined whether the alert is output a predetermined number of times or more. If the alert is output a predetermined number of times or more, the process goes to step S214, and remeasurement is started. On the other hand, if the alert is output less than the predetermined number of times, the process goes to step S206, and the processes after step S206 described above are repeatedly executed.

On the other hand, at step S210, it is determined whether it is a measurement termination time. If it is not yet the measurement termination time, the process goes to step S206, and the processes after step S206 described above are repeatedly executed. On the other hand, if it is the measurement termination time, the measurement is terminated at step S216. At step S218, the measurement data is saved, and the process is then temporarily ended.

According to this embodiment, the circadian rhythm acquisition module 311, the sleep determination module 313, and the autonomic nerve activity measurement sensor 314 have the common heart rate sensor (or pulse rate sensor) 311a. Therefore, the configuration can be simplified, and the operation can be made easier. According to this embodiment, a timing suitable for measurement can be determined to automatically perform the measurement.

### Fourth Embodiment

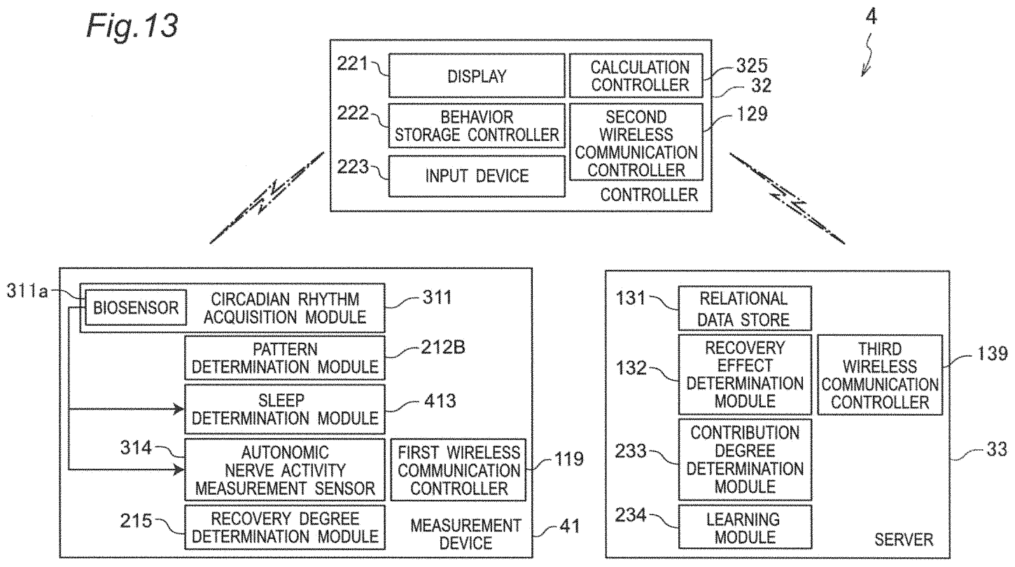

A fatigue recovery support apparatus 4 according to a fourth embodiment will be described with reference to FIG. 13. The configurations same as or similar to the third embodiment will be described in simplified manner or will not be described, and differences will mainly be described. FIG. 13 is a block diagram showing a configuration of the fatigue recovery support apparatus 4. In FIG. 13, the same or equivalent constituent elements as the third embodiment are denoted by the same reference numerals.

The fatigue recovery support apparatus 4 is different from the fatigue recovery support apparatus 3 described above in that the device includes a measurement device 41 instead of the measurement device 31. The measurement device 41 is different from the measurement device 31 described above in that the device includes a sleep determination module 413 instead of the sleep determination module 313.

The circadian rhythm acquisition module 311, the sleep determination module 413, and the autonomic nerve activity measurement sensor 314 constituting the measurement device 41 have the common biosensor 313a (heart rate sensor or pulse rate sensor). In other words, the biosensor 311a (heart rate sensor or pulse rate sensor) is shared.

The sleep determination module 413 determines a daily variation pattern of the heart rate (or pulse rate) and determines a daily variation pattern of ccvTP (or TP) obtained from the heart rate (or pulse rate). The sleep determination module 413 determines the sleep quality based on a correlation degree (inverse correlation degree) of the daily variation pattern of heart rate (or pulse rate) and the daily variation pattern of ccvTP (or TP). The TP (total power value) (msec2) is an index presenting the function of the entire autonomic nerve function and is represented by the sum of LF and HF (LF+HF). The ccvTP (%) is an index indicating the function of the autonomic nerve function. When the heart rate is high, the TP becomes high, so that the TP is corrected with the heart rate during the measurement time to obtain ccvTP.

More specifically, the sleep determination module 413 determines the daily variation pattern of heart rate (or pulse rate) through curve approximation of heart rate (or pulse wave number) data having variations based on a preset approximation rule. Similarly, the sleep determination module 413 determines the daily variation pattern of ccvTP (or TP) through curve approximation of ccvTP (or TP) data having variations based on a preset approximation rule.

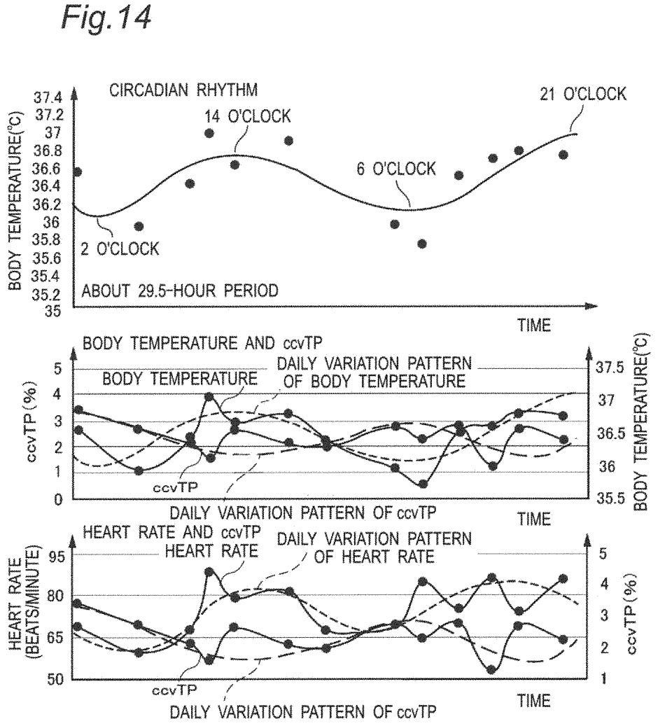

FIG. 14 shows an example of the respective daily variation patterns (correlation degree/inverse correlation degree) of body temperature and ccvTP, and an example of the respective daily variation patterns (correlation degree/inverse correlation degree) of heart rate (HB) and ccvTP. FIG. 14 shows, in order from the top, an example (of a pattern) of circadian rhythm based on a body temperature change, the example of respective daily variation patterns (correlation degree/inverse correlation degree) of body temperature and ccvTP, and the example of respective daily variation patterns (correlation degree/inverse correlation degree) of heart rate (HB) and ccvTP. The horizontal axes of FIG. 14 indicate date and time, and the vertical axes indicate body temperature (° C.), body temperature (° C.) and ccvTP, and heart rate (times/minute) and ccvTP, in order from the top. Circular plots shown in FIG. 14 indicate body temperature data, heart rate data, and ccvTP data (measured values), and approximate curves (broken lines) represent the respective patterns (daily variation patterns).

As shown in FIG. 14 (middle and bottom portions), the body temperature and ccvTP as well as the heart rate (HB) and ccvTP show daily variations having substantially reversed phases. In this regard, the inventor obtained knowledge that the correlation degree (inverse correlation degree) between the daily variation pattern of heart rate (or pulse rate, body temperature) and the daily variation pattern of ccvTP (or TP) is correlated with the sleep quality. More specifically, when the inverse correlation degree between heart rate (or pulse rate, body temperature) and ccvTP is low, the proportion of shallow sleep increases. Conversely, when the inverse correlation degree between heart rate (or pulse rate, body temperature) and ccvTP is high, the proportion of shallow sleep decreases (i.e., the proportion of deep sleep increases).

Therefore, the sleep determination module 413 obtains a correlation degree (inverse correlation degree) between the daily variation pattern of heart rate (or pulse rate) and the daily variation pattern of ccvTP (or TP), estimates a proportion of time of shallow sleep to total sleep time (sleep data) based on the correlation degree (inverse correlation degree), and determines a sleep quality based on the sleep data. The other configurations are the same as or similar to the fatigue recovery support apparatus 3 described above and therefore will not be described in detailed.

According to this embodiment, the daily variation pattern of heart rate (or pulse rate) is determined, the daily variation pattern of ccvTP (or TP) is determined, and the sleep quality is determined based on the correlation degree (inverse correlation degree) between the daily variation pattern of heart rate (or pulse rate) and the daily variation pattern of ccvTP (or TP). Therefore, the sleep quality can be determined based on quantitative data. The body temperature may be used in place of the heart rate (or pulse rate).

Although the embodiments of the present invention have been described, the present invention is not limited to the embodiments and is variously modifiable. For example, the device configurations (system configurations) are not limited to the embodiments. Therefore, although the configurations (functions) are divided into the measurement devices 11 to 31, the modules 12 to 32, and the servers 13 to 33 in the embodiments, all the configurations (functions) may be integrated, or any two (e.g., a measurement device and a module) may be integrated. Additionally, for example, the pattern determination modules 112, 212, 212B and the recovery degree determination module 215 may be included in the servers 13, 23, 33 or the modules 12, 22, 32.

### EXPLANATIONS OF LETTERS OR NUMERALS

1, 2, 2B, 3, 4 fatigue recovery support apparatus
11, 21, 21, 21B, 31, 41 measurement device
12, 22, 32 controller
13, 23, 33 server
111, 211, 311 circadian rhythm acquisition module
111a, 211a, 311a biosensor
112, 212, 212B pattern determination module
113, 313, 413 sleep determination module
113a biosensor
119 first wireless communication controller
214, 314 autonomic nerve activity measurement sensor
214a biosensor
215 recovery degree determination module
121, 221 display
222 behavior store
223 input module
129 second wireless communication controller
131 relational data store
132 recovery effect determination module
134, 234 learning module
139 third wireless communication controller
233 contribution degree determination module

1. A fatigue recovery support apparatus comprising:
a circadian rhythm acquisition module for acquiring a circadian rhythm;
one or more processors;
a pattern determination module for determining a pattern of the circadian rhythm acquired by the circadian rhythm acquisition module;
a sleep determination module for determining a sleep quality;
a relational data store for storing in advance relational data presenting a relationship among the circadian rhythm pattern, the sleep quality, and a fatigue recovery effect of sleep; and
a recover effect determination module for estimating the fatigue recovery effect of sleep based on the circadian rhythm pattern, the sleep quality, and the relational data.

2. The fatigue recovery support apparatus according to claim 1, further comprising:
an autonomic nerve activity measurement module for measuring an autonomic nerve activity index;
a recovery degree determination module for estimating a fatigue recovery degree based on a change in the autonomic nerve activity index measured by the autonomic nerve activity measurement module;
a behavior store for storing behavior information; and
a contribution degree determination module for estimating a level of contribution of sleep and behavior to the fatigue recovery degree based on the fatigue recovery degree estimated by the recovery degree determination module, the behavior information stored in the behavior store, and the fatigue recovery effect of sleep estimated by the recover effect determination module.

3. The fatigue recovery support apparatus according to claim 2, further comprising a display for presenting a sleep condition and/or a behavior suitable for fatigue recovery based on the level of contribution of sleep and behavior to the fatigue recovery degree estimated by the at least one of the processors which determine the contribution degree.

4. The fatigue recovery support apparatus according to claim 2, wherein the autonomic nerve activity measurement module detects a heart rate or a pulse rate to measure an autonomic nerve activity index indicated by any of LF/HF, LF, HF, TP, and ccvTP.

5. The fatigue recovery support apparatus according to claim 1, wherein:
the circadian rhythm acquisition module measures the circadian rhythm at least one piece of biological data among body temperature, heart rate, pulse rate, and autonomic nerve activity index; and
the pattern determination module determines the circadian rhythm pattern based on daily variation of the biological data measured by the one or more of the one or more processors that acquire the circadian rhythm.

6. The fatigue recovery support apparatus according to claim 5, wherein the sleep determination module measures at least any one piece of biological data among body motion, body temperature, heart rate, pulse rate, autonomic nerve activity index, respiratory rate, and brain wave during sleep to obtain at least any one piece of sleep data among sleep depth, duration of the each sleep depth, period, sleep time, bedtime, wake-up time, and a proportion of time of shallow sleep to total sleep time based on the biological data, and to determine the sleep quality based on the sleep data.

7. The fatigue recovery support apparatus according to claim 2, wherein the circadian rhythm acquisition module and the autonomic nerve activity measurement module are attached to a portable housing and detect a heart rate or a pulse rate to measure an autonomic nerve activity index indicated by any of LF/HF, LF, HF, TP, and ccvTP.

8. The fatigue recovery support apparatus according to claim 2, wherein the circadian rhythm acquisition module, the sleep determination module, and the autonomic nerve activity measurement module are attached to a housing wearable on a subject' s body and detect a heart rate or a pulse rate to measure the autonomic nerve activity index indicated by any of LF/HF, LF, HF, TP, and ccvTP when accepting a subject' s start operation or when automatically determining that a predetermined measurement start condition is satisfied.

9. The fatigue recovery support apparatus according to claim 1, comprising an autonomic nerve activity measurement module measuring an autonomic nerve activity index, and wherein:
the circadian rhythm acquisition module, the sleep determination module, and the autonomic nerve activity measurement module are attached to portable housing or a housing wearable on a subject' s body and detect a heart rate or a pulse rate to measure the autonomic nerve activity index indicated by any of LF/HF, LF, HF, TP, and ccvTP; and
the sleep determination module determines a daily variation pattern of heart rate or pulse rate, and a daily variation pattern of TP or ccvTP to determine the sleep quality based on a correlation degree between the daily variation pattern of heart rate or pulse rate and the daily variation pattern of TP or ccvTP.

10. The fatigue recovery support apparatus according to claim 2, wherein:
the circadian rhythm acquisition module, the sleep determination module, and the autonomic nerve activity measurement module are attached to portable housing or a housing wearable on a subject' s body and detect a heart rate or a pulse rate to measure the autonomic nerve activity index indicated by any of TP, and ccvTP; and
the sleep determination module determines a daily variation pattern of heart rate or pulse rate, and a daily variation pattern of TP or ccvTP to determine the sleep quality based on a correlation degree between the daily variation pattern of heart rate or pulse rate and the daily variation pattern of TP or ccvTP.

11. The fatigue recovery support apparatus according to claim 2, wherein the behavior store has an input accepting comments from subjects.

12. The fatigue recovery support apparatus according to claim 1, further comprising a learning module learning the subject's circadian rhythm pattern, sleep quality, and fatigue recovery degree or subject's comments acquired in the past, and reflecting a result of the learning on the relational data of the circadian rhythm pattern, the sleep quality, and the fatigue recovery effect of sleep.
